(19) United States
(12) Patent Application Publication
(10) Pub. No.: US 2018/0308584 A1
(43) Pub. Date: Oct. 25, 2018

(54) ACUTE CARE PREDICTIVE ANALYTICS TOOL

(71) Applicant: DispatchHealth Management, LLC, Denver, CO (US)

(72) Inventors: Mark H. Prather, Denver, CO (US); Kevin William Riddleberger, Superior, CO (US)

(21) Appl. No.: 15/961,695

(22) Filed: Apr. 24, 2018

Related U.S. Application Data
(60) Provisional application No. 62/488,948, filed on Apr. 24, 2017.

(51) Int. Cl. G16H 50/30; G16H 10/20; G16H 50/20; G16H 10/60

(52) U.S. Cl. CPC G16H 50/30; G16H 10/60; G16H 50/20; G16H 10/20

(57) ABSTRACT

A patient's entry into the acute care system is often under-informed or mis-informed, resulting in the patient procuring services that are not appropriate for the patient's actual needs. Systems and methods for providing right-sized acute care services can decrease cost and time-to-treatment, while maintaining quality of service for individual patients. The presently disclosed technology provides an integrated and convenient acute care triage solution that extends the capabilities of a patient's health care team. A substantial time and cost savings and resulting performance advantage may be obtained by right-sizing treatment of the patient's urgent medical condition at the patient's first point of entry into an acute care system.

### CROSS-REFERENCE TO RELATED APPLICATIONS

The present application claims benefit of priority to U.S. Provisional Patent Application No. 62/488,948 entitled “Acute Care Predictive Analytics Tool” and filed on Apr. 24, 2017, which is specifically incorporated by reference herein for all that it discloses or teaches.

### BACKGROUND

Acute care is a branch of secondary health care where a patient receives active but short-term treatment for a severe injury, severe illness, or other urgent medical condition. Acute care services are generally delivered by teams of health care professionals from a range of medical and surgical specialties. Acute care may require a stay in a hospital emergency department, ambulatory surgery center, urgent care center, or other short-term stay facility, along with the assistance of diagnostic services, surgery, or follow-up outpatient care in the community.

A patient's entry into the acute care system is often under-informed or mis-informed, resulting in the patient procuring services that are not appropriate for the patient's actual needs. More specifically, the patient may procure services that exceed that patient's actual needs, resulting in increased cost of treatment. Alternatively, the individual may procure services that are insufficient for the patient's actual needs, resulting in a transfer to a different service provider. This delays treatment for the patient and increases the associated cost of treating the patient overall.

Systems and methods for providing right-sized acute care services can decrease cost and time-to-treatment, while maintaining quality of service for individual patients.

### SUMMARY

Implementations described and claimed herein address the foregoing problems by providing a method of providing right-sized acute care services to a patient comprising collecting data from the patient, the data including identifying information and symptom information, retrieving prior health care data regarding the patient from a health information exchange using the identifying information, assigning a composite risk score to the patient based on each of the identifying information, the symptom information, and the prior health care data, and recommending an acute care service to the patient based on the assigned risk score falling within a predetermined range associated with the recommended acute care service.

Implementations described and claimed herein address the foregoing problems by further providing one or more computer-readable storage media encoding computer-executable instructions for executing on a computer system a computer process for providing right-sized acute care services to a patient, the computer process comprising the above method.

Implementations described and claimed herein address the foregoing problems by still further providing a method of providing right-sized acute care services to a patient comprising: collecting data from the patient, the data including identifying information and symptom information, retrieving prior health care data regarding the patient from a health information exchange using the identifying information, selecting one of a series of available screening protocols as a primary risk protocol based on the patient's symptom information, posing the series of questions associated with the primary risk protocol regarding the patient, wherein a composite of answers to the questions is used to assign composite risk score to the patient, assigning a composite risk score to the patient based on each of the selected primary risk protocol, answers to the series of questions, the identifying information, the symptom information, and the prior health care data, and recommending an acute care service to the patient based on the assigned risk score falling within a predetermined range associated with the recommended acute care service.

Other implementations are also described and recited herein.

### BRIEF DESCRIPTIONS OF THE DRAWINGS

FIG. 1 illustrates a first example flowchart illustrating a patient using a personalized predictive analytics tool to right-size the patient's access to acute care services.

FIG. 2 illustrates a second example flowchart illustrating a patient using a personalized predictive analytics tool to right-size the patient's access to acute care services.

FIG. 3 illustrates an example personalized predictive analytics tool using an anti-coagulation filter.

FIG. 4 illustrates an example asthma filter for a predictive analytics tool.

FIG. 5 illustrates an example patient on-boarding user interface for a predictive analytics tool.

FIG. 6 illustrates an example questionnaire for a nausea/vomiting filter selected by a user of a predictive analytics tool outputting a low-risk score.

FIG. 7 illustrates an example questionnaire for a nausea/vomiting filter selected by a user of a predictive analytics tool outputting a medium-risk score.

FIG. 8 illustrates an example questionnaire for a nausea/vomiting filter selected by a user of a predictive analytics tool outputting a high-risk score.

FIG. 9 illustrates an example dashboard for an in-queue patient of a predictive analytics tool.

FIG. 10 illustrates a general screening protocol entry form for a predictive analytics tool.

FIG. 11 illustrates a high-risk screening protocol entry form for a predictive analytics tool.

FIG. 12 illustrates example operations for a predictive analytics tool to right-size the patient's access to acute care services.

FIG. 13 illustrates an example on-scene time predictive model for a predictive analytics tool.

FIG. 14 illustrates example operations for providing right-sized acute care services to a patient.

FIG. 15 illustrates an example system diagram of a computer system suitable for implementing aspects of an acute care predictive analytics tool.

### DETAILED DESCRIPTIONS

The presently disclosed technology provides an integrated and convenient acute care triage solution that extends the capabilities of a patient's health care team.

The patient may choose from a number of options to procure acute care when presented with an injury, illness, or other urgent medical condition based on the patient's perceived needs, which may differ from the patient's actual needs. For example, the patient may call 911 to request ambulatory services, visit an emergency room (ER), visit an urgent care center, visit the patient's primary care physician's office (PCP), or call a nurse advice hotline to procure acute care. The patient's choice in selecting acute care is often under-informed and/or mis-informed (e.g., a selection is based on the patient's prior experience, prior experience(s) of a close friend or family member, results of the patient's Internet research, etc.).

For example, when a patient calls 911 and requests ambulatory services, the patient is automatically transported to an ER for treatment. No option is available for diverting the patient to a different, lower cost acute care service if ER services are not warranted for the patient's actual needs. Similarly, if the patient directly accesses an ER for treatment, the ER will diagnose and provide treatment, if needed. Any diversion of the patient to a different acute care service is subsequent to the patient's initial treatment or diagnosis at the ER, which adds cost and may delay the patient's treatment if the patient is ultimately diverted to a different acute care service.

In another example, when a patient visits an urgent care center or PCP, the patient is initially diagnosed and treated on-site. If the urgent care center or PCP does not have sufficient capability to treat the patient, the patient is referred to the ER or other acute care service. Further, some urgent care centers and PCPs lack sufficient staffing and advanced treatment capability to make any referral other than to the ER. The patient's access to the ER or other acute care service via the urgent care center or PCP may delay treatment for the patient and increase overall cost as compared to the patient accessing a right-sized acute care service directly. Further, if the patient could be sufficiently treated at the patient's PCP, but was instead treated elsewhere, treatment feedback to the patient's PCP is often inadequate or non-existent.

In yet another example, the patient may call a nurse advice hotline in an attempt to right-size their acute care service. However, the information the patient provides the nurse may be incomplete, the nurse may not have access to the patient's prior healthcare data, and the nurse does not have the ability to do any physical diagnosis or triage. In order to limit liability and due to potential use of the nurse hotline as a marketing tool, many patients may be directed to the ER when a more right-sized treatment alternative may be available.

A substantial time and cost savings and resulting performance advantage may be obtained by right-sizing treatment of the patient's urgent medical condition at the patient's first point of entry into an acute care system.

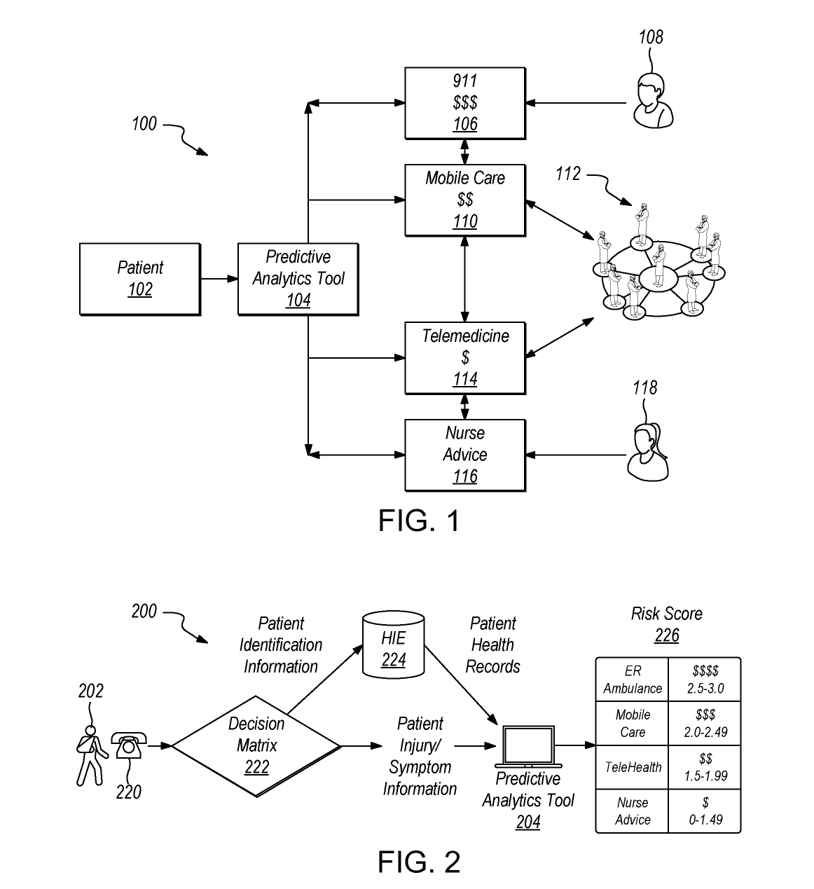

FIG. 1 illustrates a first example flowchart 100 illustrating a patient 102 using a predictive analytics tool 104 to right-size the patient's access to acute care services. The patient 102 accesses the tool 104 via a web-based interface (e.g., via a personal computer, a tablet, a smartphone, a wearable-device, etc.), a telephone-based interface (e.g., via a public switched telephone network (“PSTN”), a wireless network, a private branch exchange (“PBX”), etc.), or a combination interface (e.g., Voice over IP (“VoIP”)), which links the patient 102 to the tool 104. In other implementations, a representative for the patient 102 (e.g., the patient's medical doctor (MD), a friend, and/or an employer) may access the tool 104 on behalf of the patient 102. The tool 104 may also utilize a human representative to query the patient (or MD, friend, or employer) and input relevant data into the tool 104 on behalf of the patient 102.

The patient 102 enters identifying information and a description of the injury and/or symptoms into the tool 104. The tool 104 uses a combination of the patient' s actual medical history (e.g., pulled from a health information exchange (“HIE”), such as the Colorado Regional Health Information Organization (“CORHIO”), or other medical databases), the patient's demographics (e.g., age, sex, physical location), and the patient's description of the injury and/or symptom to risk-stratify the patient's complaint and generate a risk score to aid the patient 102 in selecting an appropriate acute care service. In implementations that include a wearable device, a camera, or other data-collecting device (not shown), the tool 104 may collect non-invasive biometric data from the patient 102 (e.g., pulse, blood pressure, imagery of an injury, etc.) for use in generating the risk score for the patient 102.

If the patient's risk score is particularly high (e.g., a score of 2.5-3.0 or “red”), the patient 102 may call 911 for ambulatory service to an ER or otherwise travel to the ER immediately (“911 $$$” 106). While ER acute care services are typically the most expensive, if the patient' s risk score is high enough, the expense is well worth it to gain access to ambulatory or ER medical personnel 108 as soon as possible.

If the patient's risk score is moderate (e.g., a score of 1.5-2.49 or “yellow”), the patient 102 may safely procure a lower cost acute care service. For example, the patient 102 may call a mobile acute care unit 110 that can at least diagnose the patient's illness or injury onsite (without transporting the patient 102 to an ER or calling an ambulatory service), and in some cases treat the patient's illness or injury onsite. In various health care environments, multiple specialized mobile acute care units may be available giving the patient 102 access to a network of medical personnel 112 larger than that available at the ER. In another example, the patient 102 may procure a telemedicine care service 114 that can also remotely diagnose the patient's illness or injury (without transporting the patient 102 to the ER) and in some cases diagnose treat the patient's illness or injury remotely. Telemedicine care service 114 can give the patient 102 access to an even larger network of medical personnel 112 physically located all over the world.

If the patient's risk score is low (e.g., 0-1.49 or “green”), the patient 102 may safely procure an even lower cost acute care service. For example, the patient 102 may call a nurse hotline (or care coordination service) 116 for guidance in treating the patient's illness or injury. More specifically, nurse 118 may review the output from the tool 104, discuss the illness or injury with the patient 102, and offer recommendations for self-treatment or other treatment of the patient's illness or injury outside of the acute care system (e.g., scheduling an appointment with the patient's PCP).

In other implementations, the tool 104 may offer additional acute care service options to the patient 102 and provide additional risk score categories. The tool 104 may also be connected to the patient's health insurance as a mechanism to pre-approve a certain level of acute care service for the patient's illness or injury to be covered by the patient's health insurance.

FIG. 2 illustrates a second example flowchart 200 illustrating a patient 202 using a personalized predictive analytics decision engine (or predictive analytics tool) 204 to right-size the patient's access to acute care services. The patient 202 may call via telephone 220 a number associated with the predictive analytics tool 204 to access a telephone-based interface and decision matrix 222, which links the patient 202 to the tool 204.

The interface and decision matrix 222 (e.g., an automated question/answer interface or a live person asking questions of the patient 202) collects two types of information from the patient 202. The first type of information is identifying information (e.g., the patient's name, date of birth, sex, social security number, driver's license number, home address, telephone number, etc.). The identifying information identifies the patient 202 to the tool 204 and allows the tool 204 to pull any available and relevant community health records on the patient 202 from an HIE 224. The HIE outputs community health records on the patient 202 that may provide input variables for the tool 204 including, but not limited to, the patient's past medical history, past surgical history, hospitalization(s), medication history, allergies, laboratory testing results, etc.

The second type of information collected from the patient 202 via the interface and decision matrix 222 is a description of the injury and/or symptoms that the patient 202 is experiencing, which may be collected via an evidence-based technology decision and data collection tree for presenting symptoms to the tool 204. A combination of the input variables from the HIE and the patient's description of the injury and/or symptoms are input into the tool 204 and the tool 204 transforms the input data into a risk score (numerical and/or visual) 226 indicating the overall urgency of the patient's illness or injury and/or a recommendation on acute care services for the patient 202. In other words, the tool 204 provides the patient 202 and/or the patient's clinical staff with data-driven care, which helps to drive the right care, at the right time, for the patient 202.

The tool 204 may use any relevant scale for scoring the urgency and/or severity of the patient's illness or injury (an overall risk factor). One example is a 3-tier scale with “Red” or “2.5-3.0” score indicates that the patient's illness or injury is severe and/or access to acute care services is urgent for the patient's well-being. A “Yellow” or “1.5-2.49” score indicates that the patient's illness or injury is significant and/or access to acute care services is semi-urgent for the patient's well-being. A “Green” or “0-1.49” score indicates that the patient's illness or injury is mild and/or access to acute care services is not urgent.

More specifically, if the patient's risk score is very high (e.g., 2.5-3.0), the tool 204 may recommend that the patient 202 immediately visit an ER or call for ambulatory service. In some implementations, the tool 204 may call 911 on behalf of the patient 202. This is typically the most expensive acute care service ($$$$) and is often handled by individual municipalities. The tool 204 may automatically reserve an ER and/or ambulance service for the highest risk scores.

If the patient's risk score is moderately high (e.g., 2.0-2.49), the tool 204 may recommend a mobile care unit for the patient 202. This is a mobile unit that has sufficient resources to come to the patient's location and treat or diagnose them on-site. In some implementations, the tool 204 may coordinate the mobile care unit on behalf of the patient 202. The mobile care unit is a lower cost option ($$$) for acute care services than an ER or ambulatory service and may provide the patient 202 with more rapid and less stressful treatment.

If the patient's risk score is moderately low (e.g., 1.5-1.99), the tool 204 may recommend a telemedicine care service (TeleHealth). The telemedicine care service can remotely diagnose the patient's illness or injury, and in some cases diagnose and/or treat the patient's illness or injury. The telemedicine care service may include telephonic interaction, secure text messaging, and/or video interaction with the patient 202, in various example implementations. The tool 204 may connect the patient 202 to the telemedicine care service directly. The telemedicine care service is a relatively low cost ($$) acute care service that may provide the patient 202 with very rapid service.

If the patient's risk score is very low (e.g., 0-1.49), the tool 204 may recommend a nurse hotline for guidance in treating the patient's illness or injury. In some implementations, the tool 204 may connect the patient 202 to the nurse hotline directly. The nurse hotline is a very low cost acute care service that may provide the patient 202 with very rapid service at a very low or zero cost ($).

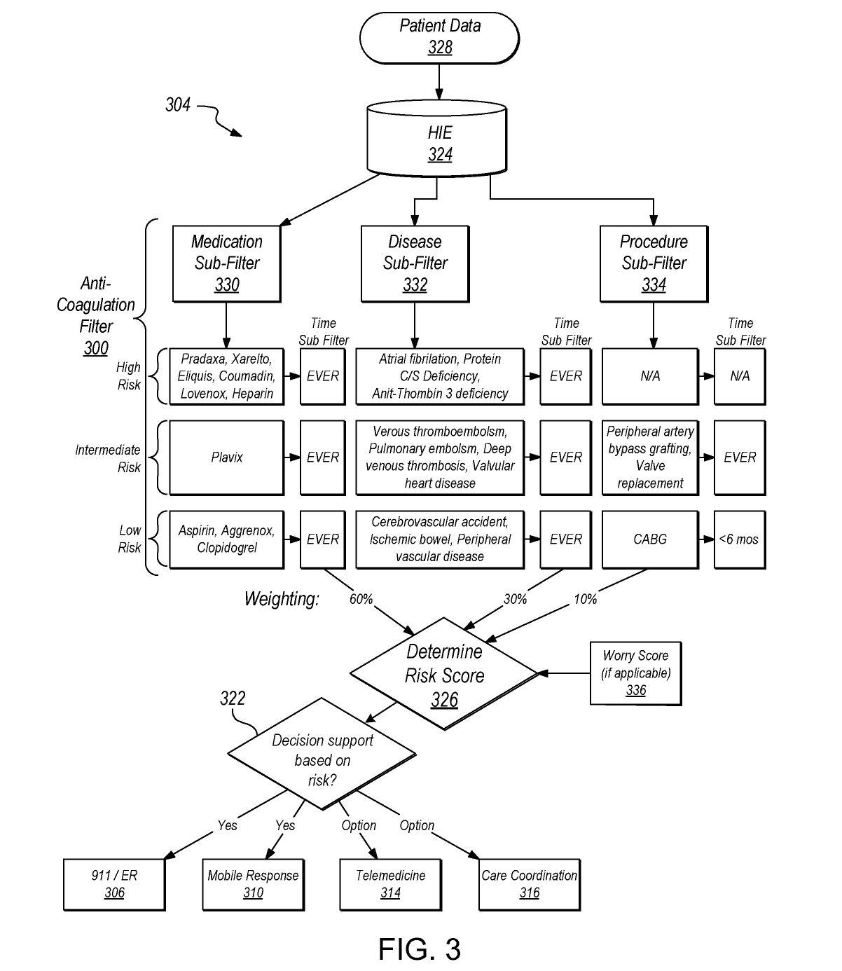

FIG. 3 illustrates an example personalized predictive analytics tool 304 using an anti-coagulation filter 300 to right-size a patient's access to acute care services. The patient enters his or her patient data 328 into an input interface (e.g., a web-browser, a touchtone telephone, voice recognition, and/or via conversation with personnel tasked with collecting the patient data 328). The patient data 328 may include the patient's name, date of birth, full or partial social security number, health insurance provider and/or coverage, gender, current physical location, home address, telephone and/or email contact information, etc.

Some or all of the patient data 328 collected at the input interface is fed into one or more applicable HIE(s) 324. The patient is identified at the HIE(s) 324 and any applicable records regarding the patient, in addition to the information received directly from the patient, are used in sub-filters 330, 332, 334 of the anti-coagulation filter 300 to determine a risk score 326 for the patient.

The medication filter 330 determines if the patient is currently or has previously consumed one or more of a listing of medications. If so, the medication filter 330 determines a time frame within which the patient has consumed the identified medications. In some implementations, the medication filter 330 will also determine a quantity of the identified medications the patient consumed. In some implementations, the medication filter 330 (or other sub-filters) will trigger the tool 304 to query the patient for additional information. For example, if an HIE record (e.g., a Systematized Nomenclature of Medicine or “SNOWMED” code) indicates that the patient has previously been prescribed Pradaxa®, but quantity or timeframe information is not available through the HIE, the tool 304 may query the patient to provide the missing information via the input interface.

The listing of medications is separated in “high risk,” “intermediate risk,” and “low risk” categories, each with a commensurate time sub-filter (here, “EVER” for each category). In other implementations, the medication filter 330 may also include a commensurate quantity sub-filter with an applicable threshold. More specifically, if the patient has ever regularly consumed “aspirin, Aggrenox®, or clopidogrel,” this is considered a “low risk” factor. If the patient has ever regularly consumed “Plavix®,” this is considered an “intermediate risk” factor. If the patient has ever regularly consumed “Pradaxa®, Xarelto®, Eliquis®, Coumadin®, Lovenox®, or heparin,” this is considered a “high risk” factor. The output of the medication filter 330 is weighted against the other sub-filters 332, 334 (here, 60% of the total) to determine the risk score 326 for the patient.

Disease filter 332 determines if the patient is currently or has previously been afflicted by one or more of a listing of diseases (e.g., via an International Classification of Diseases, 9th Revision or “ICD-9” code pulled from the HIE). An ICD-10 or later revision of the International Classification of Diseases may also be used. If so, the disease filter 332 determines a time frame within which the patient is or was afflicted by the identified diseases. The listing of diseases is separated in “high risk,” “intermediate risk,” and “low risk” categories, each with a commensurate time sub-filter (here, “EVER” for each category). More specifically, if the patient is or has ever been afflicted by a “cerebrovascular accident, ischemic bowel, or peripheral vascular disease,” this is considered a “low risk” factor. If the patient is or has ever been afflicted by a “venous thromboembolism, pulmonary embolism, deep venous thrombosis, or valvular heart disease,” this is considered an “intermediate risk” factor. If the patient is or has ever been afflicted by an “atrial fibrillation, protein C/S deficiency, or anti-thrombin 3 deficiency,” this is considered a “high risk” factor. The output of the disease filter 332 is weighted against the other sub-filters 330, 334 (here, 30% of the total) to determine the risk score 326 for the patient.

Procedure filter 334 determines if the patient has had any of a listing of procedures performed (e.g., via a Current Procedural Terminology or “CPT” code pulled from the HIE). If so, the procedure filter 334 determines a time frame within which the identified procedure(s) were performed. The listing of procedures is separated in “high risk,” “intermediate risk,” and “low risk” categories, each with a commensurate time sub-filter. Here, the “low risk” category has a commensurate “less than 6 months” time frame and the “intermediate risk” category has an “EVER” time frame. More specifically, if the patient has had a “coronary artery bypass graft surgery (CABG)” within the last 6 months, this is considered a “low risk” factor. If the patient has ever had a “peripheral artery bypass grafting or Valve replacement,” this is considered an “intermediate risk” factor. There are no “high risk” factor procedures listed in the procedure filter 334. The output of the procedure filter 334 is weighted against the other sub-filters 330, 332 (here, 10% of the total) to determine the risk score 326 for the patient.

In an example implementation, outputs from the sub-filters 330, 332, 334 are combined as follows to determine the risk score 326. Each of the sub-filters 330, 332, 334 scores the patient on a scale from 0-3. High-risk diagnoses, medications, and procedures receive a “3,” intermediate-risk diagnoses, medications, and procedures receive a “2,” low-risk diagnoses, medications, or procedures receive a “1,” and no diagnoses, medications, and procedures receive a “0.” For every diagnosis, medication, or procedure that received a risk score greater than 0, the diagnosis, medication, or procedure is run through the relevant time sub-filter. If the diagnosis, medication, or procedure occurred within the time period defined by the time sub-filter, the previously specified risk score remains. Otherwise, the previously specified risk score reverts to “0.” The resulting sub-filter risk scores are weighted and combined to yield the overall final risk score 326.

The tool 304 may be interactive (e.g., ask questions regarding the patient based on results gathered from the data input into the tool 304 from the patient and/or from the HIE) to collect the necessary information. For example, the tool 304 may output “Your patient carried a diagnosis of atrial fibrillation at one time. Confirm current anti-coagulant use.” Once the patient or health care provider confirms the patient's anti-coagulant use, the tool 304 outputs a commensurate risk score. The overall final risk score 326 (and in some implementations, the individual risk scores) are displayed to the patient and/or health care provider. In other implementations, fewer or additional rules than those described above are used to combine the outputs from the sub-filters 330, 332, 334 to determine the risk score 326.

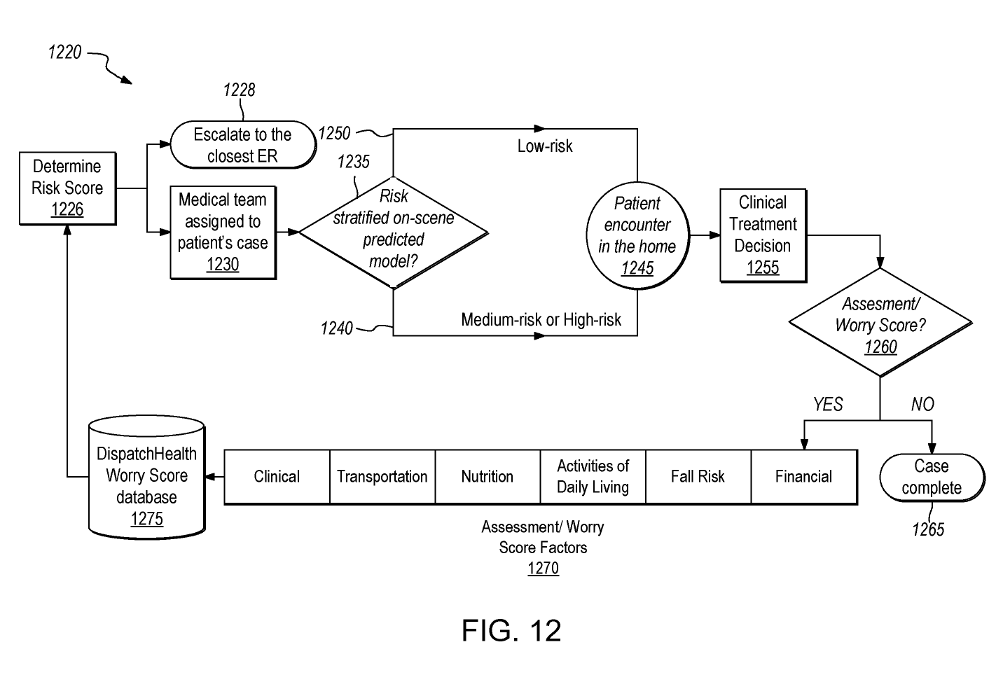

In some implementations, the risk score 326 may also incorporate an assessment or worry score 336 associated with the patient calculated using the patient's prior interactions with the tool 304. The worry score 336 is described in more detail below with reference to FIG. 12. Further, the risk score 326 may include a listing of particularly positive and/or negative factors that were primary controlling factors in determining the risk score 326. The risk score 326 is linked to a decision matrix 322 that selects or recommends one or more of a listing of available urgent care services for the patient. More specifically, “911/ER” 306, “Mobile Response Unit” 310, “Telemedicine” 314, and “Care Coordination” 316 are available to the patient. If the risk score 326 is particularly high, the decision matrix 322 may recommend that the patient receive either the 911/ER 306 services or the Mobile Response 310 services with the Telemedicine 314, and the Care Coordination” 316 services reserved for lower risk scores.

The sub-filters 330, 332, 334 are chosen specific to the anti-coagulation filter 300 from an array of sub-filters available to the predictive analytics tool 304. Other filters may use a different selection of sub-filters, time filters, and/or weighted averages. The anti-coagulation filter 300 (or another of an array of filters available to the predictive analytics tool 304) is chosen based on the patient's description of the injury and/or symptoms that the patient is experiencing.

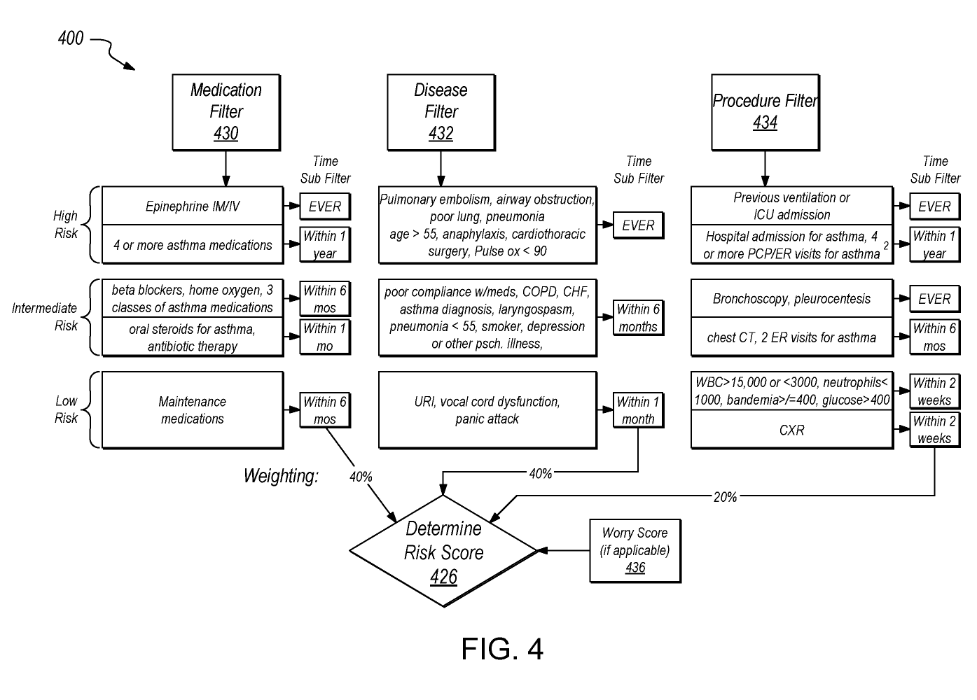

FIG. 4 illustrates an example asthma filter 400 for a predictive analytics tool to right-size a patient's access to acute care services. Filter 400 includes similar features as filter 300 of FIG. 3, with different medication, disease, and procedure listings in medication filter 430, disease filter 432, and procedure filter 434, respectively. The medication, disease, and procedure listings are separated in “high risk,” “intermediate risk,” and “low risk” categories, each with a commensurate time sub-filter.

More specifically, the medication filter 430 determines if the patient is currently or has previously taken one or more of a listing of medications. More specifically, if the patient has regularly consumed “maintenance medications” within the last 6 months, this is considered a “low risk” factor. If the patient has regularly consumed “beta blockers, home oxygen, or specific classes of asthma medications” within the last 6 months, or “oral steroids or antibiotic therapy” within the last 1 month, any of these is considered an “intermediate risk” factor. If the patient has ever regularly consumed “Epinephrine IM/IV,” or other specific classes of asthma medications” within the last year, any of these is considered a “high risk” factor. The output of the medication filter 430 is weighted against the other sub-filters 432, 434 (here, 40% of the total) to determine a risk score 426 for the patient.

The disease filter 432 determines if the patient is currently or has previously been afflicted by one or more of a listing of diseases. If so, the disease filter 432 determines a time frame within which the patient is or was afflicted by the identified diseases. More specifically, if the patient is or has been afflicted by an “upper respiratory infection, vocal cord dysfunction, or panic attacks” within in the last month, any of these is considered a “low risk” factor. If the patient is or has ever had “poor compliance with meds, chronic obstructive pulmonary disease, congestive heart failure, an asthma diagnosis, laryngitis, pneumonia (if the patient is less than 55 years of age), a smoker, suffers from depression of other psychological illness” within the last 6 months, this is considered an “intermediate risk” factor. If the patient is or has ever been afflicted by an “pulmonary embolism, airway obstruction, poor lung function, pneumonia (if the patient is greater than 55 years of age), anaphylaxis, cardiothoracic surgery, pulse oximetry less than 90” this is considered a “high risk” factor. The output of the disease filter 432 is weighted against the other sub-filters 430, 434 (here, 40% of the total) to create the risk score 426 for the patient.

The procedure filter 434 determines if the patient has had any of a listing of procedures performed. If so, the procedure filter 434 determines a time frame within which the identified procedure(s) were performed. More specifically, if the patient has had a “chest x-ray” within the last 2 weeks, this is considered a “low risk” factor. If the patient has ever had a “bronchoscopy or pleurocentesis,” a “chest computed topography scan or 2 emergency room visits related to asthma” within the last 6 months, or a “white blood cell count greater than 15,000 or less than 3,000, neutrophils less than 1000, bandemia greater than or equal to 400, or glucose greater than 400” within the last 2 weeks, any of these is considered an “intermediate risk” factor. If the patient has ever had a “previous ventilation or intensive care unit admission,” or a hospital admission for asthma or 4 or more primary care physician or emergency room visits related to asthma” within the last year, any of these is considered a “high risk” factor. The output of the procedure filter 434 is weighted against the other sub-filters 430, 432 (here, 20% of the total) to determine the risk score 426 for the patient.

In an example implementation, outputs from the sub-filters 430, 432, 434 are combined as follows to determine the risk score 426. Each of the sub-filters 430, 432, 434 scores the patient on a scale from 0-3. High-risk diagnoses, medications, or procedures receive a “3;” intermediate-risk diagnoses, medications, or procedures receive a “2;” low-risk diagnoses, medications, or procedures receive a “1;” and no diagnoses, medications, or procedures receive a “0.” For every diagnosis, medication, or procedure that received a risk score greater than 0, the diagnosis, medication, or procedure is run through the relevant time sub-filter. If the diagnosis, medication, or procedure occurred within the time period defined by the time sub-filter, the previously specified risk score remains. Otherwise, the previously specified risk score reverts to “0.” The resulting sub-filter risk scores are weighted and combined to yield the overall final risk score 426.

The associated predictive analytics tool may be interactive (e.g., ask questions regarding the patient based on results gathered from the data input into the tool from the patient and/or from the HIE) to collect the necessary information. For example, the tool may output “Your patient carried a diagnosis of atrial fibrillation at one time. Confirm current anti-coagulant use.” Once the patient or health care provider confirms the patient's anti-coagulant use, the tool outputs a commensurate risk score. The overall final risk score 426 (and in some implementations, the individual risk scores) are displayed to the patient and/or health care provider. In other implementations, fewer or additional rules than those described above are used to combine the outputs from the sub-filters 430, 432, 434 to determine the risk score 426.

The risk score 426 may also incorporate an assessment or worry score 436 associated with the patient that was calculated using the patient's prior interactions with the tool. The worry score 436 is described in more detail below with reference to FIG. 12. Further, the risk score 426 may include a listing of particularly positive and/or negative factors that were primary controlling factors in determining the risk score 426.

The sub-filters 430, 432, 434 are chosen specific to the asthma filter 400 from an array of sub-filters available to the predictive analytics tool. Other filters may use a different selection of sub-filters, time filters, and/or weighted averages. The asthma filter 400 (or another of an array of filters available to the predictive analytics tool) is chosen based on the patient's description of the injury and/or symptoms that the patient is experiencing.

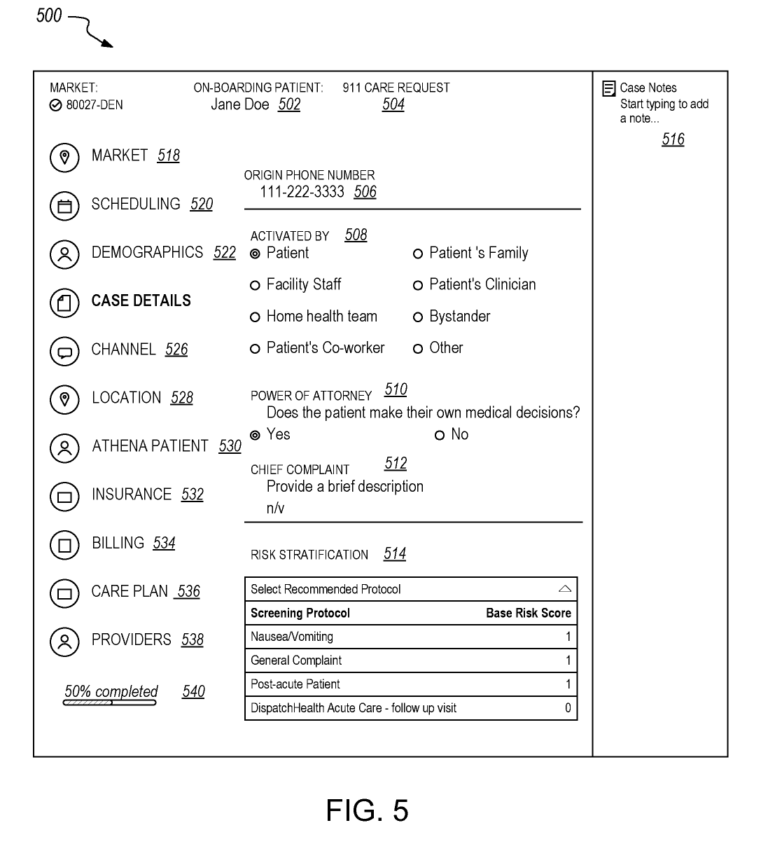

FIG. 5 illustrates an example patient on-boarding user interface 500 for a predictive analytics tool to right-size the patient's access to acute care services. In various implementations, the user interface 500 is accessed directly by a human representative (or user) for the predictive analytics tool. The human representative interacts with the patient and asks relevant questions to accurately fill out the user interface 500. In other implementations, the user interface 500 is presented directly to the patient and the patient (or user) directly inputs his/her data via the user interface 500.

The user interface 500 includes an on-boarding patient field 502 where the user (a human representative or patient) enters the patient' s name, here “Jane Doe.” A request type field 504 permits the user to enter what type of care the patient is requesting, here “911 care.” An origin phone number field 506 is either automatically populated or manually entered by the user, here “111-222-3333.” A source field 508 permits the user to identify the relation between the individual in contact with the user of the tool, here, the patient. A power of attorney field 510 permits the user to indicate whether the patient makes his/her own medical decisions, or if another individual has been granted medical power of attorney over the patient.

A chief complaint field 512 permits the user to enter words or abbreviations that indicate the patient's chief complaint, herein “n/v”, which is shorthand for “nausea/vomiting.” The tool may store and automatically present screening protocol options 514 for the chief complaint in real-time as the user enters words or abbreviations into the chief complaint field 512. In various implementations, the user may have the option to enter multiple complaints. The user also has the option to use a case notes field 516 to enter custom notes regarding the patient for later retrieval within the tool.

Additional information may be input into the tool via additional tabs accessible from the user interface 500. For example, in a market tab 518, the user enters the relevant geographic market that serves the patient's physical location where care is requested, here 80027—Denver. In a scheduling tab 520, the user is able to view the acute care services available to the user and schedule those resources appropriately according to the patient's risk score (calculated later). In a demographics tab 522, the user is able to enter demographic information (e.g., age, sex, height, weight, etc.) regarding the patient. In a channel tab 526, the user is able to enter or view the course of the patient's request for acute care services (e.g., 911, the patient's direct access, or a health care partner, such as a senior community, a home health service, a provider group, a health system, care management staff, skilled nursing facility (SNF) staff, etc.). In a location tab 528, the user enters one or more of the patient's current physical location, the patient's mailing address, and the patient's billing address. In an Athena patient tab 530, the user enters the patient's Athena ID (if applicable). In an insurance tab 532, the user enters the patient's health insurance information. In a billing tab 534, the user enters the patient's billing information (e.g., billing address, credit card information, etc.). In a care plan tab 536, the user can enter the patient's care plan (if applicable). In a Providers tab 538, the user can enter a listing of the patient's care providers. Progress bar 540 indicates the percent completion of the patient on-boarding user interface 500, here 50%.

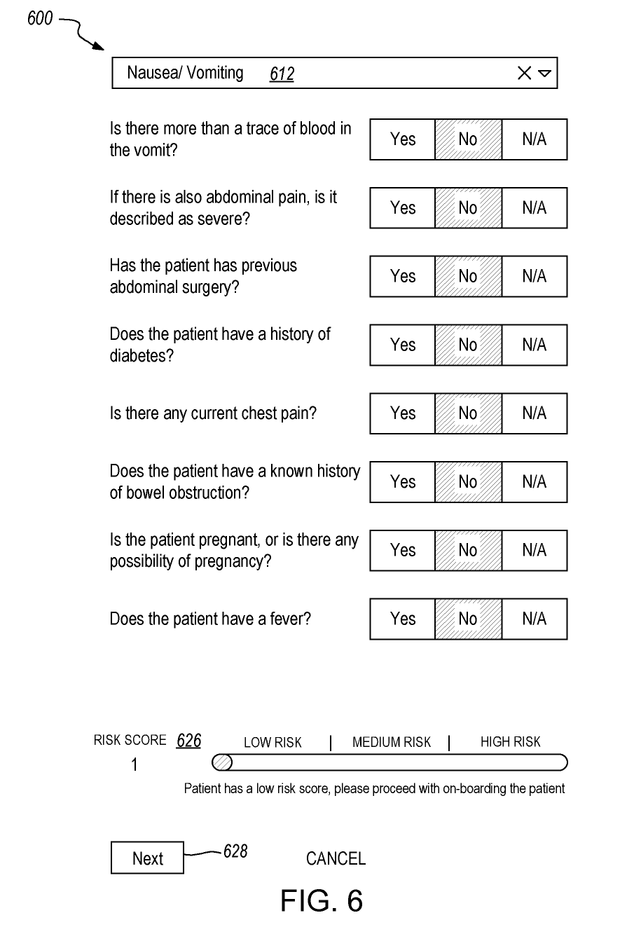

FIG. 6 illustrates an example questionnaire 600 for a nausea/vomiting filter 612 selected by a user of a predictive analytics tool outputting a low-risk score. The nausea/vomiting filter 612 may be selected from a patient on-boarding user interface, such as interface 500 of FIG. 5. The following questions are presented to the user, which the user may in turn ask the patient (or patient's representative): “Is there more than a trace of blood in the vomit?”, “If there is also abdominal pain, is it described as severe?”, “Has the patient had previous abdominal surgery?”, Does the patient have a history of diabetes?”, “Is there any current chest pain?”, “Does the patient have a known history of bowel obstruction?”, “Is the patient pregnant, or is there any possibility of pregnancy?”, and “Does the patient have a fever?”

In the example implementation of FIG. 6, the user has answered “No” to each of the questions on behalf of the patient. As a result, the predictive analytics tool has calculated a risk score 626 of “1,” which indicates a low risk to the patient. As a result, the user is instructed to proceed with the patient on-boarding process by selecting the “Next” button 628.

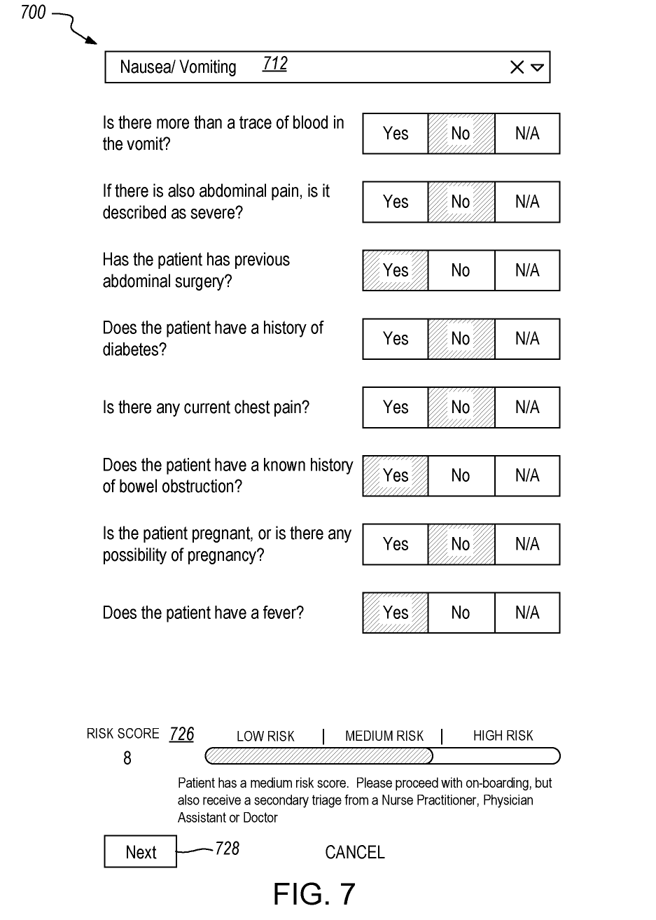

FIG. 7 illustrates an example questionnaire 700 for a nausea/vomiting filter 712 selected by a user of a predictive analytics tool outputting a medium-risk score. The nausea/vomiting filter 712 may be selected from a patient on-boarding user interface, such as interface 500 of FIG. 5. The following questions are presented to the user, which for purposes of illustration are the same as that of FIG. 6: “Is there more than a trace of blood in the vomit?”, “If there is also abdominal pain, is it described as severe?”, “Has the patient had previous abdominal surgery?”, Does the patient have a history of diabetes?”, “Is there any current chest pain?”, “Does the patient have a known history of bowel obstruction?”, “Is the patient pregnant, or is there any possibility of pregnancy?”, and “Does the patient have a fever?”

In the example implementation of FIG. 7, the user has answered “Yes” to a specific three of the questions on behalf of the patient. As a result, the predictive analytics tool has calculated a risk score 726 of “8,” which indicates a medium risk to the patient. As a result, the user is instructed to proceed with the patient on-boarding process by selecting the “Next” button 728, but also to direct the patient to receive a secondary triage from a nurse practitioner, physician assistant, or doctor.

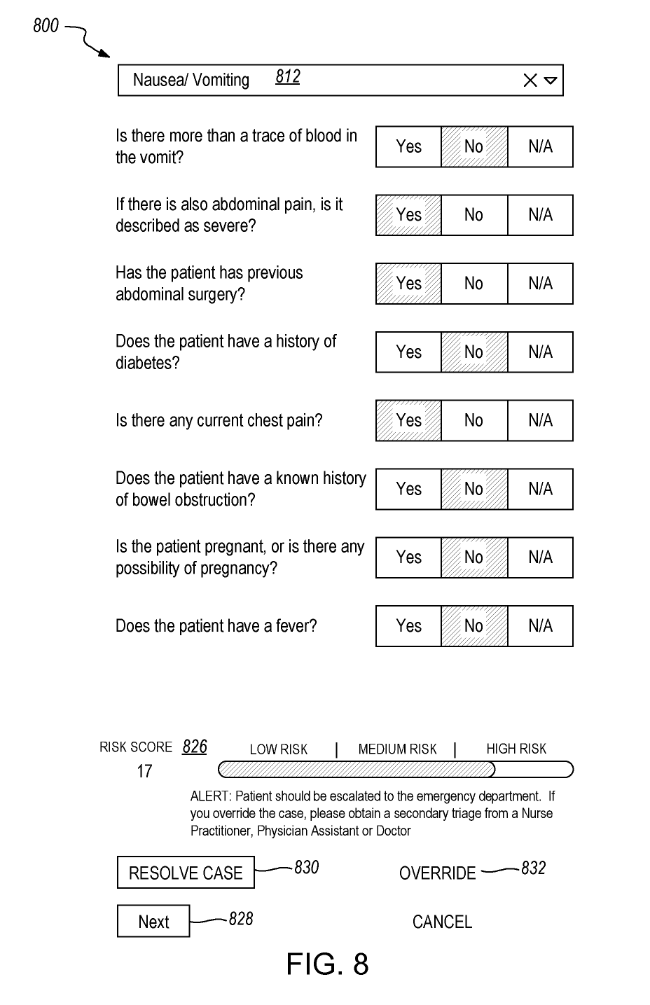

FIG. 8 illustrates an example questionnaire 800 for a nausea/vomiting filter 812 selected by a user of a predictive analytics tool outputting a high-risk score. The nausea/vomiting filter 812 may be selected from a patient on-boarding user interface, such as interface 500 of FIG. 5. The following questions are presented to the user, which for purposes of illustration are the same as that of FIGS. 6 and 7: “Is there more than a trace of blood in the vomit?”, “If there is also abdominal pain, is it described as severe?”, “Has the patient had previous abdominal surgery?”, Does the patient have a history of diabetes?”, “Is there any current chest pain?”, “Does the patient have a known history of bowel obstruction?”, “Is the patient pregnant, or is there any possibility of pregnancy?”, and “Does the patient have a fever?”

In the example implementation of FIG. 8, the user has answered “Yes” to a specific three of the questions on behalf of the patient. As a result, the predictive analytics tool has calculated a risk score 826 of “17,” which indicates a high risk to the patient. As a result, the user is instructed to escalate the patient's care to an emergency (e.g., call 911 or have the patient visit an ER) by selecting the “Resolve Case” button 830. If the user instead overrides this instruction (e.g., on instruction by the patient), the user selects the “Override” button 832, proceeds with the patient on-boarding process by selecting the “Next” button 828, and directs the patient to receive a secondary triage from a nurse practitioner, physician assistant, or doctor.

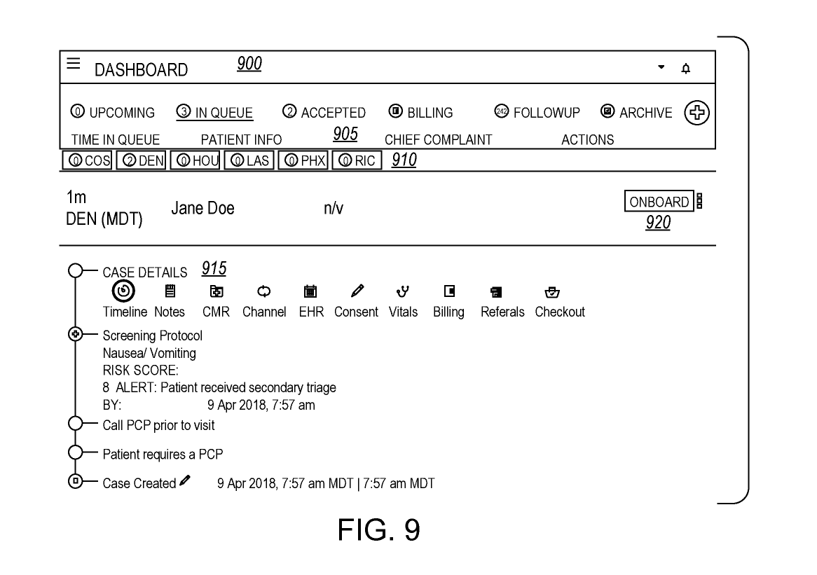

FIG. 9 illustrates an example dashboard 900 for an in-queue patient of a predictive analytics tool to right-size the patient's access to acute care services. The dashboard 900 permits a user of the predictive analytics tool to monitor a number of patients throughout their treatment experience. The dashboard 900 includes a progress bar 905, which tracks each patient as they progress through the tool. For example, “upcoming” patients have begun but not completed an intake process. “In Queue” patients have completed the intake process but have not yet been assigned an acute care solution. “Accepted” patients have been assigned an acute care solution but have not yet been treated. The “billing” tab includes billing information related to each patient using the tool. Patients in the “Follow-up” tab have been treated and are awaiting follow-up contact. The “Archive” tab includes data regarding past patients that are no longer users of the tool. Location bar 910 includes tabs associated with physical locations of acute care services offered by the tool (e.g., mobile care units). Here, the available physical locations are Colorado Springs, Colo.; Denver, Colo.; Houston, Tex.; Las Vegas, Nev.; Phoenix, Ariz.; and Richmond, Va. Other or additional locations may also be included.

An “In Queue” record for Jane Doe, including case details 915 follows below the location bar 910. The case details 915 includes a timeline, which indicates that Jane Doe's case was created by Apr. 9, 2018, that Jane Doe requires a primary care physician, that a representative of the predictive analytics tool should contact Jane Doe's primary care physician prior to rendering treatment, and that Jane Doe's chief compliant is nausea/vomiting and a risk score of “8” applies to Jane Doe's condition. Jane Doe's case details may also include notes, cardiovascular magnetic resonance imaging, channel, electronic health records, consent forms, vital readings, billing information, referrals, and a checkout process. For example, data from the HIE regarding Jane Doe, as well as her past medical history, medications, past surgical procedures, lab results, and social determinants of health may be stored in Jane Doe's case details.

In an example implementation, the Notes field of patient's case details may indicate the selected screening protocol applied to the patient (e.g., Dizziness for Jane Doe) and a time stamp that the selected screening protocol was applied. The Notes field may further indicate the patient's risk score (e.g., a score of “6”) and an indication of the type of treatment the patient has or will receive (e.g., past or upcoming secondary triage). The Notes field may still further indicate a series of questions posed to the patient, as well as the patient's responses that were used to generate the patient's risk score.

Once a user of the tool and dashboard 900 is satisfied with the information input and present in Jane Doe's case details, the user may “Onboard” 920 Jane Doe with her consent. Once Jan Does is onboard, she is placed into the “Accepted” category and will be assigned an acute care service commensurate with her condition and risk score.

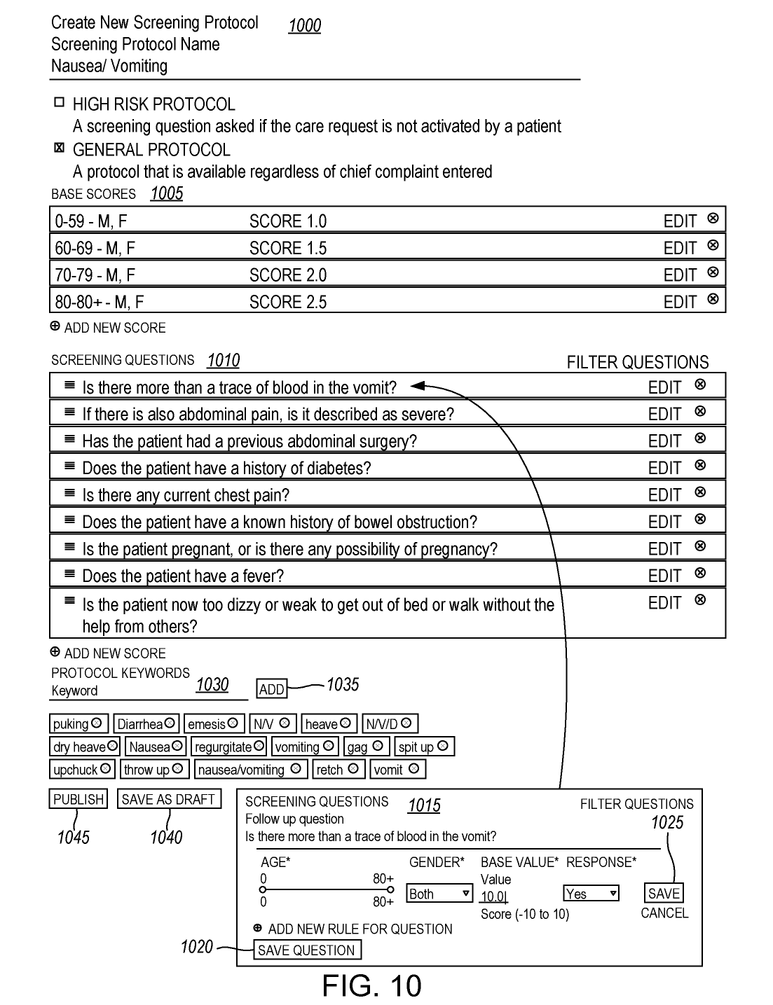

FIG. 10 illustrates a general screening protocol entry form 1000 for a predictive analytics tool. As an example, the form 1000 is being used to enter a “Nausea/Vomiting” screening protocol for the tool. Some or all other available screening protocols may also be entered into the tool using the form 1000. The screening protocol may be categorized as “high-risk” when a care request is initiated by someone other than the patient, or “general,” which is available regardless of what the patient's chief complaint is. For example, the “Nausea/Vomiting” screening protocol is categorized as a general screening protocol.

The screening protocol entry form 1000 includes a base score field 1005, which in this implementation is broken out by age group and sex. More specifically, male and female patients ages 0-59 years are assigned a 1.0 base score. Male and female patients ages 60-69 years are assigned a 1.5 base score. Male and female patients ages 70-79 years are assigned a 2.0 base score. Male and female patients ages 80+ years are assigned a 2.5 base score. In various implementations, different weightings based on age is driven by the particular protocol used by the tool (i.e., the weightings may vary dependent upon the protocol). Further, age or gender groups could have different primary weights based on the different risk protocols.

A screening questions field 1010 is made up of a series of screening questions to be posed to the patient to assess the patient's risk score based on input responses. For example, the questions posed in the screening questions field 1010 for the “Nausea/Vomiting” screening protocol include: “Is there more than a trace of blood in the vomit?”; “If there is also abdominal pain, is it described as severe?”; “Has the patient had previous abdominal surgery?”; “Does the patient have a history of diabetes?”; “Is there any current chest pain?”; “Does the patient have a known history of bowel obstruction?”; “Is the patient pregnant, or is there any possibility of pregnancy?”; “Does the patient have a fever?”; and “Is the patient now too dizzy or weak to get out of bed or walk without help of others?”. The patient's responses to the screening questions creates additional risk scores, which are combined into a composite screening risk score, which is further combined with the patient's base score to yield a final composite risk score for the “Nausea/Vomiting” screening protocol. Other screening protocols may include greater, fewer, and/or different screening questions.

Each of the screening questions have a scoring function associated therewith. For example, the “Is there more than a trace of blood in the vomit?” screening question includes the depicted scoring function 1015. Here, the scoring function 1015 applies equally to all ages and genders, but such fields may vary for other screening questions. A risk score of 10.0 is applied if the patient answers “yes” to the associated screening question. Once the user is satisfied with the screening question, the user may select the “Save Question” button 1020. Similarly, once the user is satisfied with the scoring function 1015, the user may select “Save” button 1025. Similar scoring functions apply to the other screening questions posed to the patient.

The screening protocol entry form 1000 also includes a protocol keywords field 1030, which permits the user to enter specific words that may be later used to trigger the “Nausea/Vomiting” screening protocol when a new patient is entered into the tool for screening. Here, the protocol keywords for the “Nausea/Vomiting” screening protocol include, “puking,” “diarrhea,” “emesis,” “N/V” (nausea/vomiting), “heave,” “N/V/D” (nausea/vomiting/diarrhea), “dry heave,” “nausea,” “regurgitate,” “vomiting,” “gag,” “spit up,” “upchuck,” “throw up,” “nausea/vomiting,” “retch,” and “vomit.” Other screening protocols may include greater, fewer, and/or different protocol keywords. For example, additional protocol keywords may be added using the “Add” button 1035.

If the user has partially or fully completed the “Nausea/Vomiting” screening protocol, but is not yet ready to finalize it, the user can select the “Save as Draft” button 1040, and the user (or another user) can return to the draft “Nausea/Vomiting” screening protocol later. Once the user has fully completed the “Nausea/Vomiting” screening protocol and is ready to finalize it, the user can select the “Publish” button 1045. Once published, the “Nausea/Vomiting” screening protocol is available to the tool for screening and risk scoring new patients. In various implementations, individual risk score associated with each screening question are added, averaged, weighted, or otherwise combined to create a composite screening risk score, while the base score may be added, averaged, weighted, or otherwise combined to create the final composite risk score for the “Nausea/Vomiting” screening protocol, or other screening protocols (not shown).

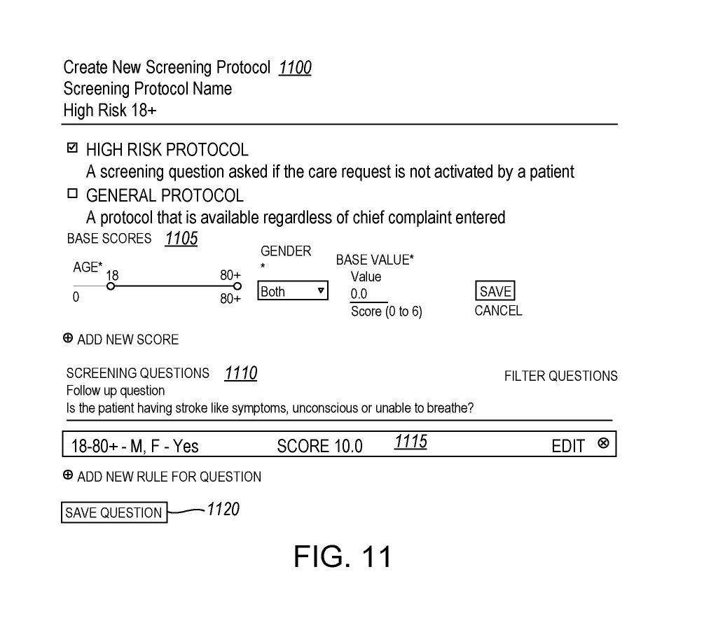

FIG. 11 illustrates a high-risk screening protocol entry form 1100 for a predictive analytics tool. As an example, the form 1100 is being used to enter a “High Risk 18+” screening protocol for the tool. Some or all other available screening protocols may also be entered into the tool using the form 1100. The screening protocol may be categorized as “high-risk” when a care request is initiated by someone other than the patient, or “general,” which is available regardless of what the patient's chief complaint is. For example, the “High Risk 18+” screening protocol is categorized as a high-risk screening protocol.

The screening protocol entry form 1100 includes a base score field 1105, which in this implementations is broken out by age group and sex. More specifically, male and female patients ages 18+ years are assigned a 0.0 base score. A screening questions field 1110 is made up of one or more screening questions to be posed to the patient to assess the patient's risk score based on input responses. For example, the question posed in the screening questions field 1110 for the “High Risk 18+” screening protocol is: “Is the patient having stroke-like symptoms, unconscious, or unable to breath?” A response to the screening question creates a risk score, which may be combined with other screening questions into a composite screening risk score, which may be further combined with the patient's base score to yield a final composite risk score for the “High Risk 18+” screening protocol. Other screening protocols may include greater, fewer, and/or different screening questions.

Each of the screening questions have a scoring function associated therewith. For example, the “”Is the patient having stroke-like symptoms, unconscious, or unable to breath?” screening question includes the depicted scoring function 1115. Here, the scoring function 1115 applies equally to all ages 18+ and genders, but such fields may vary for other screening questions. A risk score of 10.0 is applied if the patient answers “yes” to the screening question. Once the user is satisfied with the screening question, the user may select the “Save Question” button 1020. Similar scoring functions apply to other screening questions posed to the patient. The screening protocol entry form 1100 may also include protocol keywords (not shown), which permits the user to enter specific words that may be later used to trigger the “High Risk 18+” screening protocol when a new patient is entered into the tool for screening.

In various implementations, screening questions are added, averaged, weighted, or otherwise combined to create the composite screening risk score, while the base score may be added, averaged, weighted, or otherwise combined to create the final composite risk score for the “High Risk 18+” screening protocol, or other screening protocols (not shown). Other functions of the screening protocol entry form 1100 may also be similar to the screening protocol entry form 1000 of FIG. 10.

FIG. 12 illustrates example operations 1220 for a predictive analytics tool to right-size a patient's access to acute care services. A determining operation 1226 determines the patient's risk score as described in detail herein. For example, the patient's risk score may be determined pursuant to filters 300, 400 of FIGS. 3 and 4, respectively, and detailed descriptions thereof. If the patient's risk score exceeds a threshold, or qualifies as “high-risk,” the patient is immediately escalated to the closest ER 1228, either by calling an ambulatory service or instructing to the patient to immediately go to the ER.

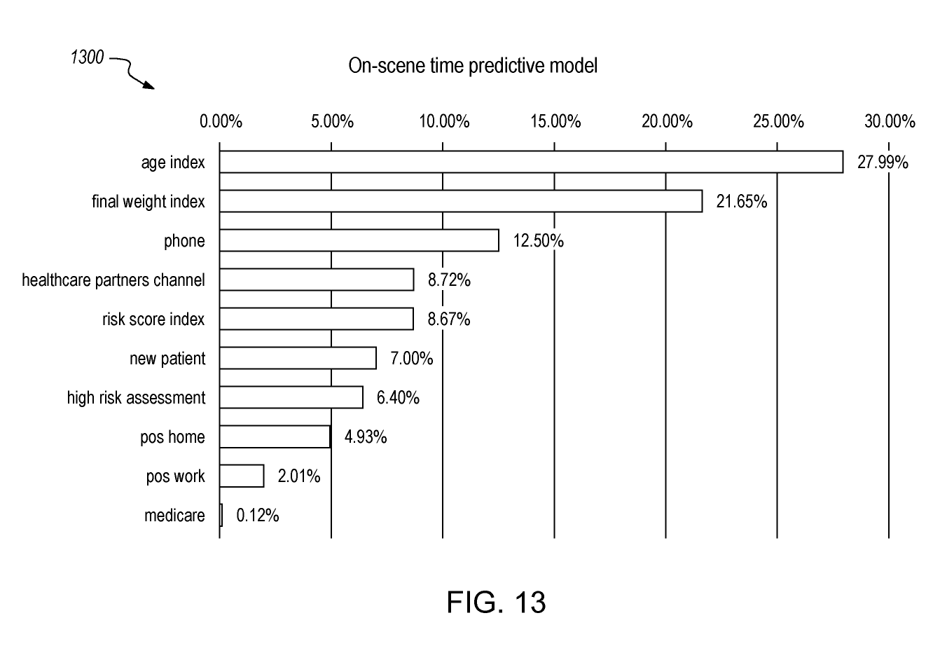

If the patient's risk score is below the threshold or qualifies as intermediate-risk or low-risk, a medical team is assigned to the patient's case 1230. The patient's position within a priority queue is then risk stratified based on an on-scene predictive model 1235. In various implementations, the on-scene predictive model 1235 is applied using a Forrest regression algorithm to discover features and relationships between on-scene time and patient-specific variables, including risk protocols. The on-scene predictive model 1235 may also take into account risk score driven predictive on-scene times (see e.g., FIG. 13 and detailed description thereof) for patients within the prioritized queue and non-prioritized queue to predict time-to-treatment for each patient awaiting treatment. While different priority queues may apply similarly to each of mobile care, telemedicine, and nurse advice acute care solutions, the remainder of FIG. 12 applies specifically to the mobile care service.

If the on-scene predictive model 1235 determines that the patient should be prioritized within the queue of patients (e.g., the patient scores as medium-risk or high-risk), the patient takes path 1240 to a patient encounter in the patient's home or other physical location 1245. In various implementations, the path 1240 may take less than 2 hours. If the on-scene predictive model 1235 determines that the patient should not be prioritized within the queue of patients (e.g., the patient scores as low-risk), the patient takes path 1250 to the patient encounter in the patient's home or other physical location 1245. In various implementations, the path 1250 may take more than 2 hours. In both scenarios, a mobile care unit visits the patient at the patient's physical location. In various implementations, the on-scene predictive model 1235 is also used to schedule and distribute workload between multiple mobile care units to achieve a time-to-service within a desired range for both prioritized patients and non-prioritized patients.

Before, during and/or after the in-home patient visit, one or more clinical treatment decisions are made 1255. Clinical guidelines and/or clinical decision support may be tied to the patient's selected risk protocol to enhance and/or standardize clinical care. Decision operation 1260 determines if the patient requires an Assessment/Worry score. The Assessment/Worry score is a gauge of the patient's ongoing medical needs that may be fed back into the risk score calculation to further fine tune the patient's risk score for future treatment. The decision 1260 is based on the patient's place of service, age, insurance, consent, partner, etc. If the patient does not require an Assessment/Worry score, the patient's case is complete 1265.

If the patient requires an Assessment/Worry score, a series of Assessment/Worry score factors 1270 are applied to determine the patient's Assessment/Worry score. Here, the factors are social determinants of health, including “Clinical,” “Transportation,” “Nutrition,” “Activities of Daily Living,” “Fall Risk,” “Social Support,” and “Financial” factors, although greater, fewer, or different factors may be applied to determine the patient's Assessment/Worry score. To assess each of the factors 1270, one or more questions are asked of the patient to gauge the patient's Assessment/Worry score.

For example, the patient (or medical care provider) may be asked if: 1) the patient had multiple ER visits or hospitalizations within the last 6 months; 2) the patient has been discharged from a hospital and/or skilled nursing facility within the last 30 days; and 3) the patient has a history of dementia, psychiatric diagnoses, myocardial infarction or coronary artery disease, stroke, diabetes, congestive heart failure, chronic obstructive pulmonary disease, or liver/renal disease to assess the patient's “Clinical” factor. The patient (or medical care provider) may also be asked if: 1) the patient has transportation to his/her medical appointments; and 2) if and what are any difficulties that the patient has getting to his/her medical appointments to assess the patient's “Transportation” factor. The patient (or medical care provider) may also be asked if: 1) the patient has access to healthy foods; and 2) if not (or only sometimes), why (e.g., trouble affording healthy foods, trouble getting to grocery store, difficulty cooking, lack of knowledge regarding nutrition, etc.)? to assess the patient's “Nutrition” factor.

The patient (or medical care provider) may also be asked if: 1) the patient has fallen within the past year; 2) if the patient feels unsteady when standing or walking; 3) if the patient worries about falling; and 4) if the patient's home potentially predisposes the patient to an increased fall risk to assess the patient's “Activities of Daily Living” factor. The patient (or medical care provider) may also be asked: 1) to assess the cleanliness of the home (e.g., clean, un-kept, in disarray, unsanitary); 2) if the patient needs help with daily activities such as bathing, preparing meals, dressing, or cleaning (if so, which activities?) to assess the patient's “Fall Risk” factor. The patient (or medical care provider) may also be asked if the patient can afford his/her medications to assess the patient's “Financial” factor.

In sum, the Assessment/Worry factors 1270 are weighted, summed, averaged, and/or otherwise combined to create an Assessment/Worry score, which is stored within an Assessment/Worry score database 1275 assessable to the tool. The patient's Assessment / Worry score is attached to the patient's medical record within the tool and may be used by the tool to calculate a future risk score for subsequent uses of the tool by the patient and to further right-size the patient's acute care needs going forward.

FIG. 13 illustrates an example on-scene time predictive model 1300 for a predictive analytics tool to right-size the patient's access to acute care services. The model 1300 graphs 10 time factors and their respective effect on on-scene time for a mobile care visit. The 10 factors are: 1) an “Age index” defined as the patient's age at the time of the patient's acute care request, scaled between 0-10; 2) a “Final weight index” defined as a sum of response weights to protocol questions, scaled between 0-10; 3) “Phone” defined as whether the acute care request was initiated by phone; 4) “Healthcare partner channel” defined as whether the acute care request was initiated by a healthcare partner; 5) a “Risk Score Index” defined as the patient's risk score, scaled between 0-10; 6) “New patient” defined as whether the patient is a new user of the tool; 7) “High risk assessment” defined as whether the patient is considered high-risk; 8) “Place of Service—Home” defined as the patient's place of service being the patient's home; 9) Place of Service—Work” defined as the patient's place of service being the patient's workplace; and 10) “Medicare” defined as whether the patient is covered under Medicare.

In an example implementation, the data for the model 1300 is pulled from all acute care requests using the tool with exceptions for incomplete requests, requests with an on-scene time of 0 minutes, requests completed in fewer than 15 minutes, including on-scene time, and requests lacking a protocol or attribute corresponding to any of the 10 monitored on-scene time factors. Other models may have greater or fewer exceptions to acute care request data.

Further, the model 1300 uses Forrest regression algorithms to discover the features and relationships between on-scene time and the 10 factors, as the variables are non-continuous and non-linear that link on-scene time to the 10 factors. The Forrest regression algorithm allows the model 1300 to estimate relationships among potential variables and predict associated weighting of the variables to arrive at an explanation of variance in on-scene time. To enhance the model 1300, a scaled index of the input variables is applied that includes an array of clinical attributes to maximize clinical variable contribution to the predicted on-scene time. As a result, the model 1300 is able to explain 85.12% of the on-scene time variance. Other models may use similar techniques but achieve greater or lesser % explanation of on-scene time variance.

The model 1300 finds that the greatest determinant of on-scene time variance is the “Age Index,” which contributes to 28% of on-scene time variance. More specifically, older patients require significantly more on-scene time than younger patients, even taking into account older patients being over represented in the model 1300 as compared to younger patients. Other factors % contribution to on-scene time variance are also shown in the model 1300. The model 1300 allows the tool to accurately predict on-scene time for individual patients based on patient-specific data. This allows the on-scene predictive model 1235 of FIG. 12, for example, to be more accurate.

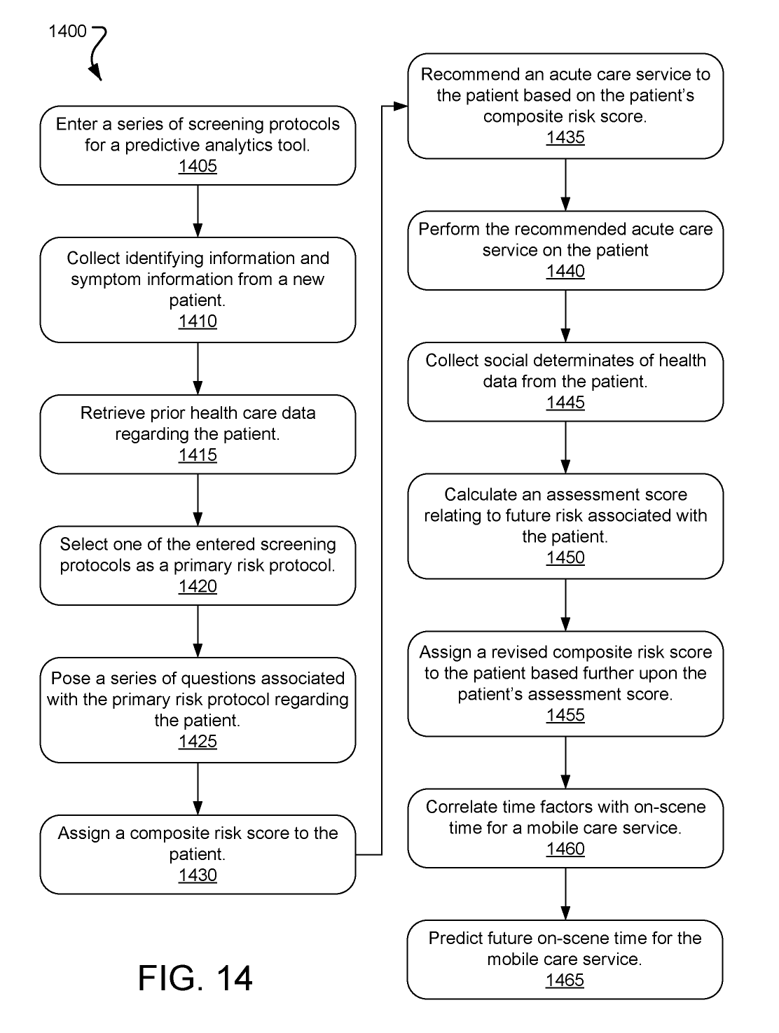

FIG. 14 illustrates example operations 1400 for providing right-sized acute care services to a patient. In an entering operation 1405, a user enters a series of screening protocols, each defined by a base score and a series of questions to be posed regarding the patient. The screening protocols each define a potential primary risk protocol to be used to generate a risk score associated with the patient upon entry for a predictive analytics tool. In a collecting operation 1410, a user (the same or a different user from operation 1405) collects data from a new patient, the data including identifying information and symptom information. The identifying information is associated specifically with the patient's identity, demographics, location, etc., while the symptom information is associated specifically with the patient's condition that has triggered the patient to request acute care using the tool.

A retrieving operation 1415 retrieves prior health care data regarding the patient from a health information exchange using the patient's identifying information. The retrieving operation 1415 may pull information from any available health care database. A selecting operation 1420 selects one of the entered screening protocols as a primary risk protocol based on the patient's symptom information. In various implementations, keywords entered during the collecting operation 1410 regarding the patient's symptoms is compared against keywords associated with each available screening protocol. A user selects the most appropriate available screening protocol as the primary risk protocol.

A posing operation 1425 poses a series of questions associated with the primary risk protocol regarding the patient. In various implementations, an individual risk score is calculated for each answer of each of the questions. Further, time filters may be applied to each of the questions. An assigning operation 1430 assigns a composite risk score to the patient based on the selected primary risk protocol, answers to the series of questions, the identifying information, the symptom information, and the prior health care data. In some implementations, the composite risk score is a combination of the individual risk scores calculated from each of the answers collected during the posing operation 1425, combined with a base score associated with the patient.

A recommending operation 1435 recommends an acute care service to the patient based on the assigned risk score falling within a predetermined range associated with the recommended acute care service. In various implementations, the available options for a recommended acute care service include an ER visit, a visit from a mobile care unit, a telemedicine service, and a nurse advice line. As an example, the highest risk score range is assigned to the ER visit, a medium-high risk score range is assigned to the mobile care unit, a medium-low risk score range is assigned to the telemedicine service, and a low risk score range is assigned to the nurse advice line.

In a performing operation 1440, a medical care provider performs the recommended acute care service on the patient. In a collecting operation 1445, the medical care provider or another user of the predictive analytics tool collects social determinants of health data from the patient following the performed acute care service. Using the collected social determinants of health, a calculating operation 1450 calculates an assessment score relating to future risk associated with the patient. For future interactions with the patient (e.g., when the patient uses the tool to receive future acute care services), in an assigning operation 1455, the tool assigns a revised composite risk score to the patient based further upon the patient's calculated assessment score.

In a correlating operation 1460, the tool correlates one or more time factors, each associated with multiple patients, to on-scene time for the mobile care service. In a predicting operation 1465, the tool predicts future on-scene time for the mobile care service based on the correlated time factors.

The embodiments of the invention described herein are implemented as logical steps in one or more computer systems. The logical operations of the present invention are implemented (1) as a sequence of processor-implemented steps executing in one or more computer systems and (2) as interconnected machine or circuit modules within one or more computer systems. The implementation is a matter of choice, dependent on the performance requirements of the computer system implementing the invention. Accordingly, the logical operations making up the embodiments of the invention described herein are referred to variously as operations, steps, objects, or modules. Furthermore, it should be understood that logical operations may be performed in any order, unless explicitly claimed otherwise or a specific order is inherently necessitated by the claim language.

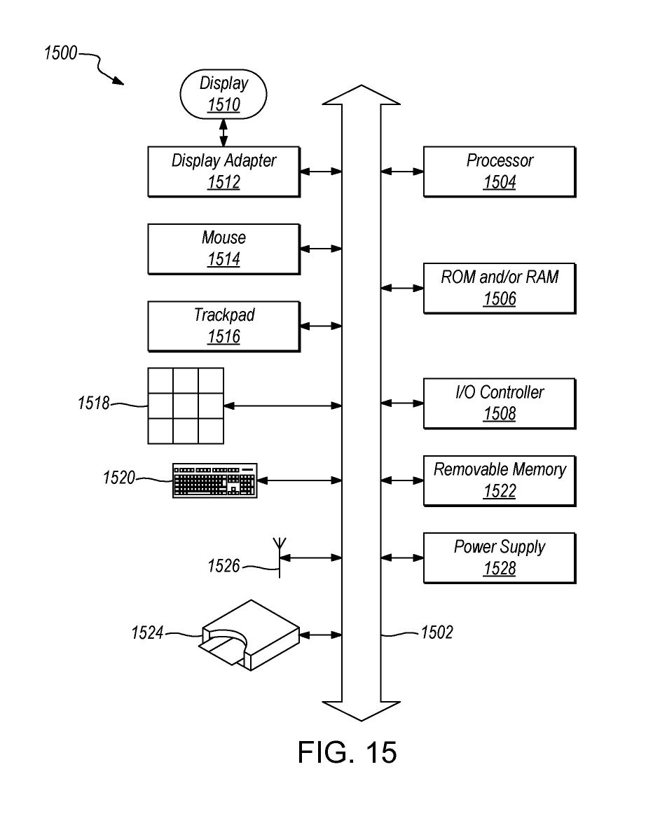

FIG. 15 illustrates an example system diagram of a computer system 1500 suitable for implementing aspects of the predictive analytics tool. System 1500 includes a bus 1502 which interconnects major subsystems such as a processor 1504, internal memory 1506 (such as RAM and/or ROM), an input/output (I/O) controller 1508, removable memory (such as a memory card) 1522, and external devices such as display screen 1510 via display adapter 1512, a mouse 1514, a trackpad 1516, a numeric keypad 1518, an alphanumeric keyboard 1520, a smart card adapter or acceptance device 1524, a wireless antennae or other interface 1526, and a power supply 1528. Many other devices can be connected. Wireless interface 1526 together with a wired network interface (not shown), may be used to interface to a local or wide area network (such as the Internet) using any network interface system known to those skilled in the art.

Many other devices or subsystems (not shown) may be connected in a similar manner (e.g., servers, personal computers, tablet computers, smart phones, mobile devices, etc.). Also, it is not necessary for all of the components depicted in FIG. 15 to be present to practice the presently disclosed technology. Furthermore, devices and components thereof may be interconnected in different ways from that shown in FIG. 15. Code to implement the presently disclosed technology may be operably disposed in the internal memory 1506 or stored on storage media such as the removable memory 1522, a thumb drive, a CompactFlash® storage device, a DVD-R (“Digital Versatile Disc” or “Digital Video Disc” recordable), a DVD-ROM (“Digital Versatile Disc” or “Digital Video Disc” read-only memory), a CD-R (Compact Disc-Recordable), or a CD-ROM (Compact Disc read-only memory). For example, in an implementation of the computer system 1500, code for implementing the predictive analytics tool described in detail above may be stored in the internal memory 1506 and configured to be operated by the processor 1504.

Aspects of the predictive analytics tool may be implemented in a tangible computer-readable storage media readable by a computer. The term “tangible computer-readable storage media” includes, but is not limited to, random access memory (“RAM”), ROM, EEPROM, flash memory or other memory technology, CDROM, digital versatile disks (DVD) or other optical disk storage, magnetic cassettes, magnetic tape, magnetic disk storage or other magnetic storage devices, or any other tangible medium which can be used to store the desired information and which can be accessed by mobile device or computer. In contrast to tangible computer-readable storage media, intangible computer-readable communication signals may embody computer readable instructions, data structures, program modules, or other data resident in a modulated data signal, such as a carrier wave or other signal transport mechanism.

The above specification, examples, and data provide a complete description of the structure and use of exemplary embodiments of the invention. Since many embodiments of the invention can be made without departing from the spirit and scope of the invention, the invention resides in the claims hereinafter appended. Furthermore, structural features of the different embodiments may be combined in yet another embodiment without departing from the recited claims.

What is claimed is:

1. A method of providing right-sized acute care services to a patient comprising:
collecting data from the patient, the data including identifying information and symptom information;
retrieving prior health care data regarding the patient from a health information exchange using the identifying information;
assigning a composite risk score to the patient based on each of the identifying information, the symptom information, and the prior health care data; and
recommending an acute care service to the patient based on the assigned composite risk score falling within a predetermined range associated with the recommended acute care service.

2. The method of claim 1, wherein the prior health care data includes one or more of the patient's prior medications, diseases, and performed procedures.

3. The method of claim 2, wherein assigning the composite risk score includes:
calculating individual risk scores associated with each of the patient's prior medications, diseases, and performed procedures; and
combining the individual risk scores to generate the composite risk score.

4. The method of claim 3, further comprising:
applying time filters to each individual risk score; and
changing the individual risk scores if the patient's prior medications, diseases, and performed procedures are outside a time frame specified by the time filters.

5. The method of claim 2, further comprising:
applying a time filter to the composite risk score; and
changing the composite risk score if the patient's prior medications, diseases, and performed procedures are outside a time frame specified by the time filter.

6. The method of claim 1, further comprising:
entering a series of screening protocols, each defined by a base score and a series of questions to be posed regarding the patient.

7. The method of claim 6, further comprising:
selecting one of the entered series of screening protocols as a primary risk protocol based on the patient's symptom information, wherein the assigned composite risk score is associated with the selected primary risk protocol.

8. The method of claim 7, further comprising:
posing the series of questions associated with the primary risk protocol regarding the patient, wherein a composite of answers to the questions is used to assign the composite risk score to the patient.

9. The method of claim 1, wherein the acute care service is selected from one of at least an emergency room visit, a visit from a mobile care unit, a telemedicine service, and a nurse advice line, each assigned to a different predetermined range of risk score.

10. The method of claim 1, further comprising:
performing the recommended acute care service on the patient;
collecting social determinants of health data from the patient following the performed acute care service;
calculating an assessment score relating to future risk associated with the patient; and
assigning a revised composite risk score to the patient based further upon the patient's calculated assessment score.

11. The method of claim 1, wherein the recommended acute care service is a mobile care service, further comprising:
correlating one or more time factors, each associated with multiple patients, to on-scene time for the mobile care service; and
predicting future on-scene time for the mobile care service based on the correlated time factors.

12. One or more computer-readable storage media encoding computer-executable instructions for executing on a computer system a computer process for providing right-sized acute care services to a patient, the computer process comprising:
collecting data from the patient, the data including identifying information and symptom information;
retrieving prior health care data regarding the patient from a health information exchange using the identifying information;
assigning a composite risk score to the patient based on each of the identifying information, the symptom information, and the prior health care data; and
recommending an acute care service to the patient based on the assigned composite risk score falling within a predetermined range associated with the recommended acute care service.

13. The computer-readable storage media of claim 12, wherein assigning the risk score includes:
calculating individual risk scores associated with each of the patient's prior medications, diseases, and performed procedures; and
combining the individual risk scores to generate the composite risk score.

14. The computer-readable storage media of claim 12, wherein the computer process further comprises:
entering a series of screening protocols, each defined by a base score and a series of questions to be posed regarding the patient.

15. The computer-readable storage media of claim 14, wherein the computer process further comprises:
selecting one of the entered series of screening protocols as a primary risk protocol based on the patient's symptom information, wherein the assigned composite risk score is associated with the selected primary risk protocol.

16. The computer-readable storage media of claim 15, wherein the computer process further comprises:
posing the series of questions associated with the primary risk protocol regarding the patient, wherein a composite of answers to the questions is used to assign composite risk score to the patient.

17. The computer-readable storage media of claim 12, wherein the acute care service is selected from one of at least an emergency room visit, a visit from a mobile care unit, a telemedicine service, and a nurse advice line, each assigned to a different predetermined range of risk score.

18. The computer-readable storage media of claim 12, wherein the computer process further comprises:
performing the recommended acute care service on the patient;
collecting social determinants of health data from the patient following the performed acute care service;
calculating an assessment score relating to future risk associated with the patient; and
assigning a revised composite risk score to the patient based further upon the patient's calculated assessment score.

19. The computer-readable storage media of claim 12, wherein the recommended acute care service is a mobile care service, wherein the computer process further comprises:
correlating one or more time factors, each associated with multiple patients, to on-scene time for the mobile care service; and
predicting future on-scene time for the mobile care service based on the correlated time factors.

20. A method of providing right-sized acute care services to a patient comprising:
collecting data from the patient, the data including identifying information and symptom information;
retrieving prior health care data regarding the patient from a health information exchange using the identifying information;
selecting one of a series of available screening protocols as a primary risk protocol based on the symptom information;
posing a series of questions associated with the primary risk protocol regarding the patient;
assigning a composite risk score to the patient based on each of the selected primary risk protocol, answers to the series of questions, the identifying information, the symptom information, and the prior health care data; and
recommending an acute care service to the patient based on the assigned composite risk score falling within a predetermined range associated with the recommended acute care service.
